(19) United States
(12) Patent Application Publication
(10) Pub. No.: US 2007/0270802 A1
(43) Pub. Date: Nov. 22, 2007

(54) Device and a method for joining, fixing and aligning broken bones and dislocated joints of vertebrates

(76) Inventor: Pranjal Sharad Kodkani, Mumbai (IN)

Correspondence Address: John Dodds, 1707 N St. NW, Washington, DC, 20036, US

(21) Appl. No.: 11/429,342

(22) Filed: May 5, 2006

(51) Int. Cl. A61B 17/00

(52) U.S. Cl. 606/59

(57) ABSTRACT

A new device and a method for joining, fixing and aligning broken bones and dislocated joint of vertebrates is disclosed. A preferred embodiment of the device comprises a uniplanar-bilateral frame for stable fixation of dome osteomy of upper end of tibia. The device enables positioning of orthopedic pins either in parallel or in a desired angle in relation to each other. The device according to this disclosure allows corrections and adjustments during and after surgery.

### TECHNICAL FIELD OF THE INVENTION

The present invention relates to a device and a method for joining, fixing and aligning broken bones and dislocated joints of vertebrates. More particularly this invention relates to a device to be used by medical surgeons for the fixation of the dome osteotomy of the upper end of tibia of Homo sapiens.

### BACKGROUND OF THE INVENTION

When the bone structure of humans is either fractured and or osteotomised (surgically cut) on account of an accident, deformity at birth or on account of old age it becomes necessary that certain bones or joints need to be fixed and or aligned when such incidents occur. In order to join and or align these bones or joints, in certain cases it becomes essential for the medical practitioner to provide external support. For providing external support to the damaged, fractured and/or osteotomised bone or dislocated joints the patient is required to undergo surgery by which external objects such as bolts, clamps, pins, screws, plates and or rods of metal are embedded into the bone structure capable of aiding in joining and or aligning the damaged, fractured and/or osteotomised bone or dislocated joints.

Depending on the use and shape of the bone or joint required to be repaired, external objects are introduced. In the case when tibia bone in the human body is broken on account of osteotomy thin pins are introduced in the body for supporting the external device known as a fixator. A fixator is a device used by various medical surgeons to aid in the joining and aligning of different bones of vertebrates.

Prior to the present invention whenever there was a need for the medical practitioners, especially the orthopedic surgeons, to make a surgical cut in the bone in the shape of a dome it would be necessary for them to support and stabilize the bone or joint by the use of fixators. A surgical cut in the bone, in the shape of a dome is known as dome osteotomy. Hence, when dome osteotomy of the upper end of tibia is done in vertebrate and especially in Homo sapiens, fixators of different types are used. Tibia is the larger inner bone situated in the lower limbs of the vertebrates between knee and ankle. Hence, to stabilize dome osteotomy of the upper end of tibia external fixators are used for joining and aligning the same. The tibia enables vertebrates and especially humans to walk and supports the weight of the upper half of the body.

Fixators are of various types but can be broadly classified into three distinct groups based on the construction and application of the fixator namely: (i) unilateral fixators (like the Orthofix), (ii) the uniplanar bilateral fixator (like the Charnleys) and (iii) the multiplanar fixator (Ilizarov).

The unilateral fixator is a device which is affixed only to one side of the bone for aiding in the stabilization thereof. This device is held onto the bone by using pins or screws embedded into the bone via surgery. Though regularly used by orthopedic surgeons, the unilateral fixators are found to be unstable, especially in cases of dome osteotomy where it is better that the bone held by fixators should be from both of the sides rather than being held or supported from one side only. This fixator being capable of supporting the bone from one side only causes delay in the recovery and or mobilization of the patient. The lack of stability further causes the surgeon not to have the desired control over the dome osteotomy as the one sided apparatus causes a deviation in the alignment of the osteotomy becasue the maximum effect and weight of the device is always on one side and not balanced.

One of the uses of the fixator to be used in dome osteotomy of the upper end of tibia is also to aid in the tibia to support the weight of the patient which could vary from a few kilograms to 100 s of kilograms. Having the device on one side only also causes the unilateral fixator, on several occasions to be incapable of sturdily holding the weight of the patient and at the same time securely holding osteotomy in the desired position and place.

On account of lack of stability of the unilateral fixator the uniplanar bilateral fixator was introduced. The uniplanar bilateral fixator, besides having common features of fixator such as using of clamps to hold onto the pins and screws, it has a bilateral design i.e. it provides support to the bone from both sides. This device has been preferred by several orthopedic surgeons as having the capability of stabilizing the dome osteotomy and also the weight of the patient. However, the stability by the uniplanar bilateral fixator, is not without its share of troubles as it over or under corrects the deformity. The inaccuracy in correcting the deformity by the uniplanar bilateral fixator is because of the mandatory parallel placement of the proximal and distal pins, which are situated opposite to each other. The positioning of the proximal and distal pins causes the clasping and holding of the dome osteotomy to be held in place and under the desired pressure for healing and aligning of the osteotomy. The use of this fixator prevents settings or adjustments at desired angles both at the time of surgery and thereafter because the pins are always to be parallel.

Many times there is a need for the surgeon to correct and or adjust the settings of the fixator post operative as on weight bearing a further change in the osteotomy alignment and or desired pressure may be required. The impossibility or difficulty in achieving the desired angle of corrections and or adjustments of the osteotomy would cause deformity or cause a compromise in the results.

The multiplanar fixator (Ilizarov) is also a fixator like the uniplanar bilateral fixator, which besides having common features such as clamps, pins and or screws has multiple metal rings with fenestrations, which are assembled together with long threaded rods. The ring construction encircles the limb from all the sides and is fixed to the underlying bone with long wires. The wires can be passed in multiple directions (i.e. planes) to affix the bones with the rings and therefore this fixator is known as a multiplanar fixator. This fixator is the most commonly used fixator. On account of having several planes in which the bone can be affixed it allows the surgeon to be able to achieve the desired angles at which the alignment or setting of the bone or joint is required. Also the multiplanar construction of the fixator enables stabilization as it surrounds the bone of the limb from all sides. However, the use of the multiplanar fixator has disadvantages too, namely its value, size, weight, appearance and social/patient acceptability.

The multiplanar fixator is the most stable fixator available in the market currently and popular among the surgeons. The fixator requires every procedure and technical detail to be followed which is very time consuming and needs the surgeon to be present during all the procedures. Use of the multiplannar fixator has its own effect on the patient, socially and otherwise: i) the fixator is more expensive ii) the surgery requires more time, thereby causing more money to be spent on the surgeons and medication iii) the fixator itself is very heavy and cumbersome to move with, and iv) the alteration in garments is required by the patient in order to move around with the fixator. The changes and or readjustments required for the patient to undergo when using the multiplanar fixator is not only expensive but also emotionally stressing.

An object of present invention is to provide a very efficient and improved fixator which permits the orthopedic surgeon to be able to affix the fixator in short period of time, which is small and compact and at the same time achieve accurate and precise results by simultaneously providing good stability to the osteotomy.

Another object of this fixator is to permit the surgeon to not only to be able to do adjustment and correct the desired angle and pressure at the osteotomy with the fixator during the operation but also easily thereafter.

It is also one of the objects of this fixator to be capable of having a stable fixation for the dome osteotomy.

Another object is that the fixator according to this disclosure is patient friendly, sleek, light weight and economical and easy to use.

It is also one of its primary objectives to cause the patient to recover faster, thereby causing early and easy movement or mobilization and also be able to independently support the patient's weight.

The above and other objects of this invention are achieved by the new fixator which has been designed and devised to overcome the existing problems and difficulties faced by both the medical practitioners and the patients while using the most popular fixators discussed above.

A preferred embodiment of the fixator of the present invention comprises a uniplanar-bilateral frame for stable fixation of the dome osteotomy of the upper end of tibia, while it simultaneously enables positioning of the orthopedic pins either in parallel or in a desired angle in relation to each other.

The fixator is also capable of corrections and adjustments to be made during and after surgery and is user friendly having sleek body and light weight.

Accordingly, the new fixator, is a device having a design which is suitable for stabilizing and aligning the dome (barrel-vault) osteotomy of the upper end of tibia and to overcome the drawbacks of other fixators and to provide certain major advantages over them.

Accordingly, present invention provides device for joining, fixing and aligning broken bones and dislocated joints of vertebrates. The devise comprises two threaded rods each having at distant pair of movable clamps capable of being secured on the rod at a desired position; each clamp comprising a sliding block and a swiveling block, said swiveling block being mounted on top of the sliding block by means of riveting; said sliding block further being a cube and having two pairs of opposite vertical sides, a through hole being drilled through one set of the vertical sides, thereby providing a tunnel through the sliding block for inserting said threaded rod through it. In a preferred embodiment the diameter of the tunnel is slightly larger than the diameter of the rod, whereby the clamp slides along the rod. The clamp can be secured at a desired position on the rod by lock nuts that are locating at both side of the sliding block. The second set of opposite vertical sides of said sliding block is having threaded holes for inserting locking bolts for securing the swivel block on the top of the sliding block. Said swivel block is cubical in shape and has two sets of opposite vertical sides and further having two tunnels drilled through each sets of vertical opposite sides. The tunnels are used for receiving an orthopedic pin (e.g. Denham/Steinmann pin). The top side of said swivel block has a threaded hole for receiving a locking bolt to secure said Denham/Steinmann pin. The clamps positioned at one end of the rods are used to receive and secure an orthopedic pin that intrudes the bone at one location and the clamps positioned at the other end of the rods are used to secure another pin that intrudes the bone at another location. By sliding the clamps along the rod a desired angle can be adjusted for the pins and the clamps can thereafter be fixed to keep the desired angle.

Now the invention is described with reference to the accompanying drawing

### BRIEF DESCRIPTION OF THE DRAWINGS

FIG. 1a—Diagrammatic representation of fixator clamp secured on a rod and its parts.
(a) Bolt for securing Steinmann/Denham pin
(b) Swivel block.
(c) Sliding block.
(d) Nut on threaded rod for securing the position of the sliding block (locking nut).
(e) Threaded Rod
(f) Tunnel for Denham/Steinmann pin.
(g) Locking bolt.

FIG. 1b—Diagrammatic transverse section through a clamp.

FIG. 1c—Diagrammatic vertical section through a clamp.

FIG. 1d—Diagrammatic presentation of two clamps attached to the rod.

FIG. 2a—Proximal Denham and distal Steinmann pin at x degrees to each other. Dome osteotomy performed above tibial tuberosity.

FIG. 2b—Distal fragment rotated within the osteotomy till both pins are parallel to each other. The pins are secured with the clamps on the rods.

FIG. 3a—Under correction (assessed post op): Aligning of the bones is corrected by distracting one end of the pins by moving the clamps along the rod until the desired degree of correction is achieved.

FIG. 3b—Compression is done on both sides simultaneously to re-impact the osteotomy by sliding each pair of clamps closer to each other along the rods.

FIG. 3c—Final alignment is achieved.

### SUMMARY OF THE INVENTION

According to a preferred embodiment of this invention the new fixator and all its components are made of materials such as steel or carbon fiber, but not limited to these. The new fixator is a uniplanar-bilateral fixator having 2 threaded rods with 8-12 nuts, 8 blocks (4 sliding and 4 swivel blocks), fixed with 2 orthopedic pins such as Steinmann pins/Denham pins. Steinmann/Denham pins may be made of steel or carbon fiber and used for fixing into the bone for aiding in the affixation of the fixator.

The threaded rod can vary in thickness from 2 mm to 20 mm in diameter depending on the size and type of the bone requiring to be stabilized and also the weight of the patient. Similarly, the length may also vary depending on the shape, length and placement of the bones or joints requiring the support of the fixator and therefore it may vary from 1 inch to 3 meters. On each threaded rod, two clamps are placed, one on each end. The clamps consist of a set of blocks having a bigger block capable of sliding motion and a smaller block capable of swivel movement. The blocks are identified by the movement/function/motion that they perform (i.e. sliding block and swivel block).

The bigger block, called the sliding block is a cube preferably having a ratio of 1:1:1. Depending upon the size and type of the joint or bone to be stabilized the dimensions of the block may vary. The base of the sliding block is flat, while the 4 vertical sides have holes. Two holes on opposite sides of the block are used to pass the threaded rod through it. The holes have a diameter which is slightly larger than the diameter of the threaded rod, preferably by a fraction of a millimeter. This difference in diameter aids the sliding motion required for the functioning of the threaded rod. The other two holes are threaded so that in each of them there is a locking bolt, which is capable of securing the rivets with which the smaller block is attached on top of the sliding block.

The smaller block is called the swivel block and it is placed on the top of the sliding block and secured there by a rivet. The dimensions of the swivel block may vary preferably in a manner that the diagonal length of the swivel block is equivalent to the length of the sliding block There are tunnels in the swivel block which receive and aid in securing the Steinmann pins/Denham pins. The pins are held in position by means of a bolt secured onto the top of the swivel block. Preferably the bolt is of a length such that not more than 3 threads are permitted to appear in the tunnel to secure in place the pins therein. The tunnels are made in a manner so that it is possible to accommodate different sizes of pins. Securing of the pins in the tunnel with the bolt does not prevent the swiveling movement of the block. The swiveling movement of the block is controlled by the nuts on both sides of the sliding block. Tightening and or loosening of the nuts on the sliding block is required for firmly securing the rivet of the swivel block to be held in place and incapable of free movement. Tightening of the bolts of the sliding block does not interfere with the sliding mechanism of the sliding block.

On preventing the motion of the swivel block as aforesaid, the two blocks that are the sliding and swivel block become one unit or clamp and are therefore capable of the sliding motion only. The ability of the swivel block to slide, at the post operative stage, over the threaded rod permits the alteration of pressure between the two pins and or altering the distance between them, thereby enabling the surgeon to correct and or adjust the position/pressure of osteotomy. The position of the clamps is maintained by securing the nuts on the threaded rods on both sides of the clamps.

In order to achieve and maintain the precise degree of correction it becomes essential for the orthopedic surgeon to use a combination of pins which may be placed parallel to each other or maybe at an angle in relation to each other. The new fixator enables the surgeon to either adjust the pins parallel to each other or at an angle in relation to each another. This is of great importance and aid, especially during recovery when the angles of the pins and pressure on the osteotomised bone have to be varied.

The present invention further provides for the method of correcting and or readjusting the alignment of the bone at the osteotomy in the immediate post operative stage, without further surgery.

Another aspect of this invention is to provide for a fixator capable of a sliding and swivel movement.

Use of the invented clamp is provided in dome osteotomy surgery of the upper end tibia of humans and it comprises of the following steps:
a) After the patients deformity is diagnosed the angle of correction required to set the deformity right is calculated by using set principles limb alignment.
b) Then the patient undergoes surgery wherein the proximal pin is introduced into the upper end of the tibia, while the distal pin is introduced at a lower level and at an angle to the proximal pin.
c) Subsequently the dome osteotomy is made between the space of the two pins on the upper end of the tibia.
d) The bone is then rotated within the dome to be placed and positioned to the required alignment.
e) When the bone is at the desired position the clamps of the fixator are fixed to the pins.
f) Even if the preferred embodiment of the instant invention is to use the fixator as a biplanar fixator it would be clear to one skilled in the art that the same idea can be used as unilateral fixator i.e. only one rod and two clamps would be involved.

### DETAILED DESCRIPTION OF THE INVENTION

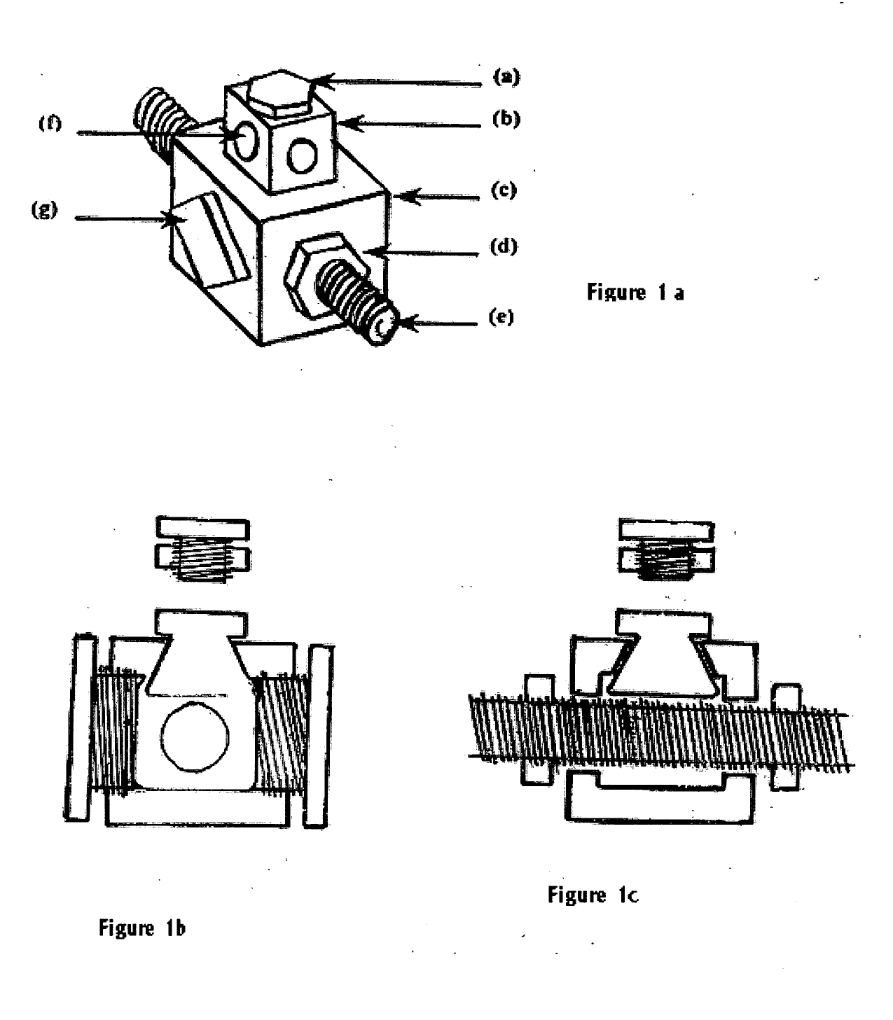

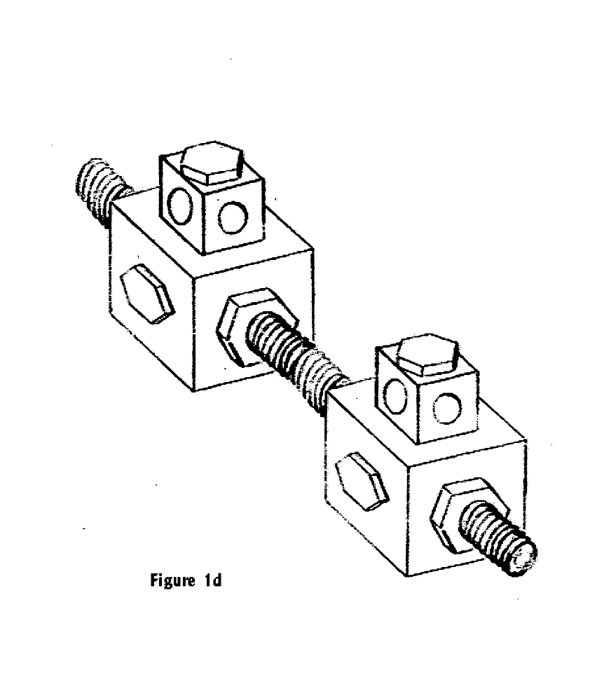

The present invention now will be described in details, with reference to FIGS. 1 to 3. The fixator comprises two pairs of clamps (4 clamps). Each clamp is separate. Each clamp has two blocks, namely a sliding block (Shown in FIG. 1a as ‘c’) and a swivel block (shown in FIG. 1a as ‘b’). Each pair of clamps is kept at distance and mounted onto one threaded rod (FIG. 1a ‘e’) and each clamp is locked with a pair of nuts from both sides as shown in FIG. 2b. Each clamp comprises a sliding block having a cubic shape and thereby two sets of vertical sides. A tunnel is drilled through the block though one set of vertical sides. The block is mounted onto the threaded rod by passing it through the tunnel. The block is secured/held in its place by the locknuts (FIG. 1a‘d’). The other set of vertical sides have threaded holes for attachment of a locking bolt (FIG. 1a ‘g’) On top side of the sliding block a hole is drilled to fix on it the swivel block (FIG. 1a ‘b’) by means of a rivet. The shape of the swivel block is rectangular having four vertical sides through which tunnel (FIG. 1a ‘f’) is drilled for inserting the Denham/Steinmann pin. The top of the swivel block is plane and has in its centre a threaded hole for placement of a securing the bolt (FIG. 1a ‘a’) therein so that the pins i.e. Denham/Steinmann are secured and/or held in place.

FIG. 1b and 1c show the structure of the clamps and FIG. 1d shows two clamps secured to the rod.

The new fixator is a uniplanar bilateral fixator which requires the pins to be inserted on side of the bone and fixed to the pins with the bolt on the swivel block. The nuts placed over the threaded rods keeps the 2 pins at desired distance and compressed against each other. The correction achieved is confirmed radiographically and with a bovie cord from the center of the hip to the center of the talus. Once this is done, new fixator is fixed into a static position by additionally tightening the locking bolts on the sliding block.

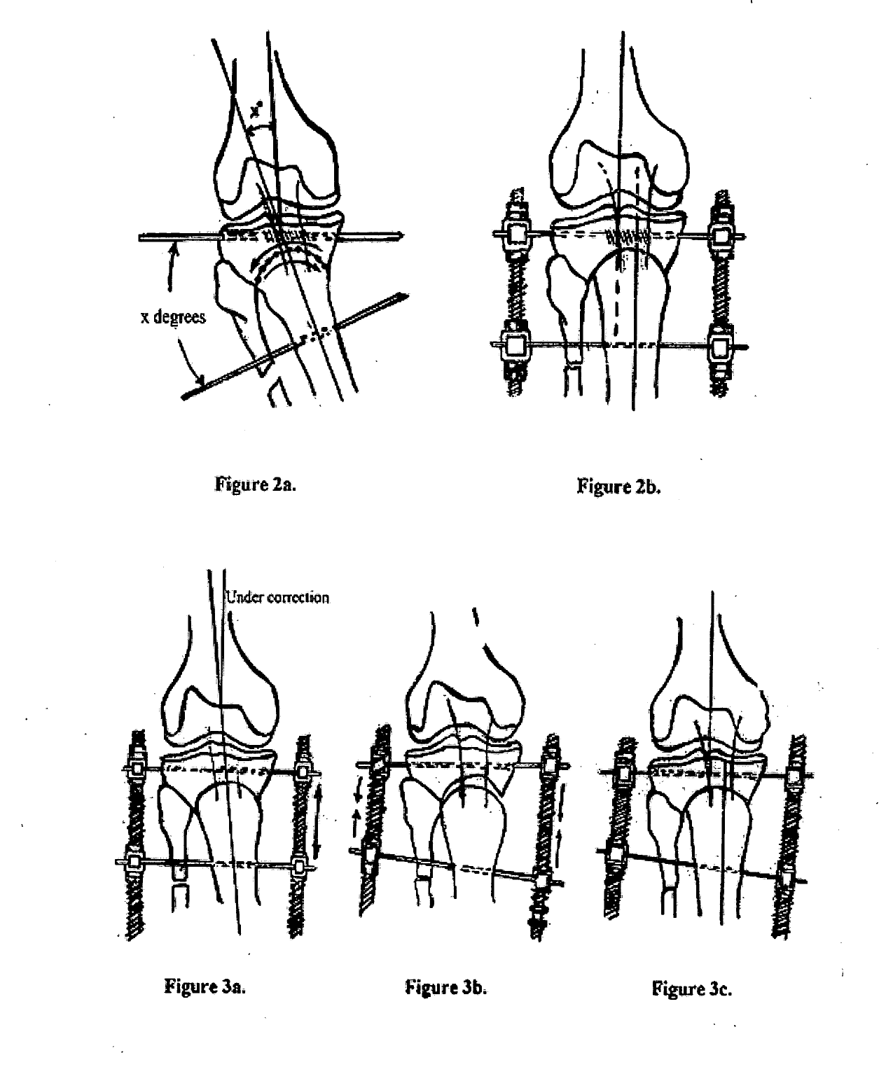

Alteration correction is done as follows and shown in FIG. 3:

Step 1:

Make sure all swivel blocks are unlocked (locking nuts are kept loose).

Distraction is done on the respective side to disimpact at the osteotomy site and at the same time achieve the required degree of correction.

Step 2:

Compression is done at both sides simultaneously to reimpact at the osteotomy site. The frame is then made static by locking the clamps with the locking nuts.

The dome osteotomy has good inherent stability in the anteroposterior plane owing to the broad metaphysical region and the confining nature of the osteotomy. The fixator due to its bilateral frame provides mediolateral stability, with additional stability given by compression at the osteotomy. Besides the fixator and the structure of the osteotomy which provide majority of the stability, the soft tissues around the osteotomy add on to its stability, similarly as they stabilize the knee joint. These are, the patellar tendon anteriorly, the medial collateral ligament medially, the Popliteus and Gastrocnemius posteriorly.

The typical examples given below to describe the invention do not limit the compositional range covered under the spirit and scope of the invention.

### EXAMPLE

Use of invention is illustrated with reference to following figures.

FIG. 2a—Proximal Denham and distal Steinmann pin at x degrees to each other. Dome osteotomy is performed above tibial tuberosity.

FIG. 2b shows distal fragment rotated within the osteotomy till both pins are parallel to each other.

FIG. 3a shows under correction (assessed post op) alignment is corrected by distracting the pins from one end by moving the clamps on one rod till the desired degree of correction is achieved.

FIG. 3b shows compression is done on both sides simultaneously to reimpact the osteotomy by moving the clamps on both of the rods closer to each other.

FIG. 3c shows final alignment achieved.

This invention may, however, be embodied in many different forms and should not be construed as limited to the embodiments set forth herein; rather, these embodiments are provided so that this disclosure will be thorough and complete, and will fully convey the scope of the invention to those skilled in the art.

I claim:

1. A device for joining, fixing and aligning broken bones and dislocated joints of vertebrates, said device further comprising:
two threaded rods;
four clamps and;
two orthopedic pins;
said threaded rods having two ends, and
one clamp being movably positioned in each end of each rod,
and said pins having first and second ends, further penetrating vertebrate bone and being affixed from their first ends to the clamps locating at the ends of one rod and from their second ends to the clamps locating in the ends of another rod; and
said clamps further comprising a sliding block, a swivel block and two locknuts,
said sliding block being cubic and having two pairs of vertical sides and a top and a bottom side, a through hole being drilled through the first set of vertical sides thereby forming a tunnel through the sliding block and the threaded rod passing through the sliding block via the tunnel,
said locknuts being positioned on the rod on both sides of the sliding block so as to secure the clamp at a desired position on the threaded rod,
said swiveling block being cubic and having two pairs of vertical sides and a top and a bottom side and said swiveling block being attached on the top side of the sliding block by means of riveting,
two through holes being drilled through both sets of vertical sides of the swiveling block, thereby forming two tunnels for securing one end of one orthopedic pin, and said top side of the swiveling block further having a threaded hole for placing a securing bolt therein, said securing bolt securing the end of the pin passing through the tunnel, and
said swiveling block further being secured to the sliding block with locking bolts placed in threaded holes drilled in the second set of vertical sides of the sliding block;
and the pins being capable of locating parallel or in an angle in relation to each other depending on the position of the clamps on the rods.

2. The device of claim 1, wherein the clamps and/or the rod are made of steel.

3. The device of claim 1, wherein the clamps and/or the rod are made of carbon fiber.

4. The device of claim 1, wherein the diameter of the tunnel in the sliding block is by a portion of millimeter larger than the diameter of the rod passing the through the tunnel.

5. The device according to claim 1, wherein the swiveling block is such that it allows use of different sizes and types of pins.

6. A method for joining, fixing and aligning broken bones and dislocated joints of vertebrates, said method comprising the steps of:
a) introducing a proximal pin into an upper end of tibia and a distal pin at a lower level of tibia;
b) providing osteomy in between the space of the proximal and distal pins;
c) aligning the bone in desired position;
d) fixing the pins with a fixator according to claim 1; and
e) and optionally post operationally correcting the alignment of the bones by adjusting the pins by moving the clamps of claim 1.
